(19) United States
(12) Patent Application Publication
(10) Pub. No.: US 2008/0294060 A1
(43) Pub. Date: Nov. 27, 2008

(54) DEVICES AND METHODS FOR DISEASE DETECTION, MONITORING AND/OR MANAGEMENT

(75) Inventors: Carlos F. Haro, St. Paul, MN (US); Kent Lee, Shoreview, MN (US); Kenneth C. Beck, St. Paul, MN (US); Yi Zhang, Blaine, MN (US); Jeffrey E. Stahmann, Ramsey, MN (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 11/751,111

(22) Filed: May 21, 2007

(51) Int. Cl. A61B 5/087

(52) U.S. Cl. 600/538

(57) ABSTRACT

Embodiments of the invention are related to methods and devices for respiratory or cardiac disease detection, monitoring, and/or management. In an embodiment, the invention includes a method of calculating a pulmonary function parameter of a subject. The method can include obtaining a first signal indicative of lung volume change during breathing from a first sensor, obtaining a second signal indicative of distending pressure from a second sensor, and calculating the pulmonary function parameter based on the first signal and the second signal. In an embodiment, the invention includes a method of monitoring pulmonary or cardiac disease status. In an embodiment, the invention includes an implantable medical device. The implantable medical device can include a first sensor configured to produce a first signal indicative of lung volume change during breathing, a second sensor configured to produce a second signal indicative of intrapleural pressure, and a processor configured to calculate lung compliance or pulmonary resistance based on the first signal and the second signal. Other aspects and embodiments are provided herein.

### TECHNICAL FIELD

This disclosure relates generally to devices and methods for disease detection, monitoring, and/or management. More specifically, the disclosure relates to implantable medical devices and related methods for respiratory and/or cardiac disease detection, monitoring, or management.

### BACKGROUND OF THE INVENTION

Pulmonary diseases afflict millions of people each year. In addition, pulmonary diseases remain a leading cause of death in the United States. Pulmonary diseases are frequently associated with other types of diseases, such as diseases of the heart (cardiac disease). Cardiac disease can have a detrimental effect on lung function, both with and without co-existing lung disease.

Monitoring patients' physiological state is an important aspect in the diagnosis, management and treatment of pulmonary and cardiac diseases and related conditions. For this reason, significant efforts have been directed at improving monitoring and detection technologies for pulmonary and cardiac diseases.

Unfortunately, many current techniques for monitoring pulmonary function can only be performed in a care facility, such as a hospital or a clinic. This is because such techniques generally rely on the use of specialized equipment that requires training and knowledge to be safely and properly used. For this reason, many current techniques for monitoring or detecting pulmonary and cardiac diseases generally only provide sporadic snap-shots of a patient's condition, which can hinder early detection of symptoms and identification of adverse trends.

For at least these reasons, a need exists for additional methods of gathering pulmonary data regarding a patient. A need also exists for methods of detecting, monitoring, and/or managing pulmonary or cardiac diseases and conditions.

### SUMMARY OF THE INVENTION

Embodiments of the invention are related to methods and devices for respiratory and/or cardiac disease detection, monitoring, and/or management. In an embodiment, the invention includes a method of determining a pulmonary function parameter of a subject. The method can include obtaining a first signal indicative of lung volume change during breathing from a first sensor and obtaining a second signal indicative of distending pressure of the lungs from a second sensor, wherein at least one of the first and second sensors is chronically implanted. The method can also include calculating the pulmonary function parameter based on the first signal and the second signal.

In an embodiment, the invention includes a method of monitoring pulmonary or cardiac disease status. The method can include obtaining a first signal indicative of lung volume change during breathing with a first sensor and obtaining a second signal indicative of distending pressure of the lungs with a second sensor, wherein at least one of the first and second sensors is chronically implanted. The method can also include calculating a pulmonary function parameter based on the first signal and the second signal and trending the value over a period of time.

In an embodiment, the invention includes an implantable medical device. The implantable medical device can include a first sensor configured to produce a first signal indicative of lung volume change during breathing and a second sensor configured to produce a second signal indicative of intrapleural pressure. The device can also include a processor configured to calculate a pulmonary function parameter based on the first signal and the second signal.

In an embodiment, the invention includes a method of titrating drug therapy. The method can include obtaining a first signal indicative of lung volume change during breathing from a first sensor and obtaining a second signal indicative of distending pressure from a second sensor, wherein at least one of the first and second sensors is chronically implanted. The method can include calculating a value of a pulmonary function parameter based on the first signal and the second signal. The method can also include comparing the pulmonary function parameter with a baseline pulmonary function parameter and adjusting drug therapy if indicated based on the comparison.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a graph of hypothetical volume-pressure curves for emphysema (curve A), normal lungs (curve B), and pulmonary fibrosis (curve C).

FIG. 2 is a schematic view of components of the respiratory system.

FIG. 3 is a cross-sectional top schematic view of the heart and parts of the pulmonary artery in a human.

FIG. 4 is a graph of an idealized pulmonary artery pressure signal.

FIG. 5 is a schematic view of an implanted medical device including a pressure sensor in the intrapleural space in accordance with an embodiment of the invention.

FIG. 6 is a schematic view of an implanted medical device including a pressure sensor and a volume sensor coupled to a single lead in accordance with an embodiment of the invention.

FIG. 7 depicts a pressure-time plot and a volume-time plot as used in a method for calculating pulmonary resistance.

FIG. 8 is a pressure-volume plot for a healthy lung illustrating both inspiration and expiration.

FIG. 9 is a diagram of components of an implantable medical device in accordance with an embodiment of the invention.

FIG. 10 is a flowchart of a method for monitoring pulmonary parameter trends.

FIG. 11 is a hypothetical graph of lung compliance over time illustrating changes common in pulmonary edema.

FIG. 12 is a hypothetical graph of lung compliance over time illustrating changes common in pulmonary fibrosis.

FIG. 13 is a hypothetical graph of lung compliance over time illustrating changes common in an asthma attack.

FIG. 14 is a hypothetical graph of lung compliance over time illustrating changes common in emphysema.

FIG. 15 is a flow chart illustrating a method for titrating drug therapy.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Monitoring a patient's pulmonary status is an important aspect in the diagnosis, management and treatment of various pulmonary and/or cardiac diseases. One relevant pulmonary function parameter is lung compliance. Lung compliance (C_{L}) refers to how readily the lungs accept a volume of inspired air. Lung compliance (C_{L}) is defined as the change in lung volume (ΔV) per unit pressure change (ΔP). Generally, lung compliance is expressed in units of liters per centimeter of water pressure (L/cm H₂O).

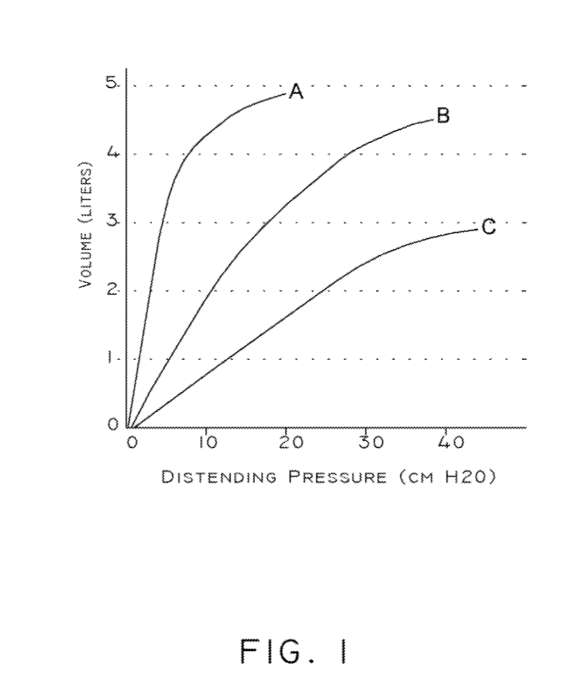

Lung compliance can be a diagnostic parameter for some types of pulmonary or cardiac diseases or conditions. For example, when a patient suffers from pulmonary fibrosis, the lungs become stiff, increasing the distending pressure necessary to increase the volume of air within the lungs (inspire air). Fibrotic lungs would be considered poorly compliant. In contrast, emphysema, a condition where many alveolar walls are lost, results in the lungs becoming so pliant that only a small distending pressure is necessary to create a large volume change. Thus, the lungs in emphysema would be considered highly compliant. By way of example, FIG. 1 shows a graph of hypothetical expiratory volume-pressure curves for emphysema (curve A), normal lungs (curve B), and pulmonary fibrosis (curve C). Lung compliance is the slope of these curves. As can be seen in FIG. 1, lung compliance can distinguish the two diseases states (curves A and C) from the normal state (curve B).

Lung compliance can be measured as static lung compliance, quasi-static lung compliance, or dynamic lung compliance. The distinction between static and dynamic lung compliance is that static lung compliance is unaffected by resistive effects of airflow on pressure. As such, for static compliance, measurements of pressure and volume must be taken in the absence of airflow. Of course, as a practical matter, living subjects must continue to breath and thus airflow can only be interrupted briefly. As such the term “quasi-static” is sometimes used to describe measurements taken during brief interruptions to airflow that are intended to approximate static compliance. As used herein, the terms “static” and “quasi-static” are used interchangeably.

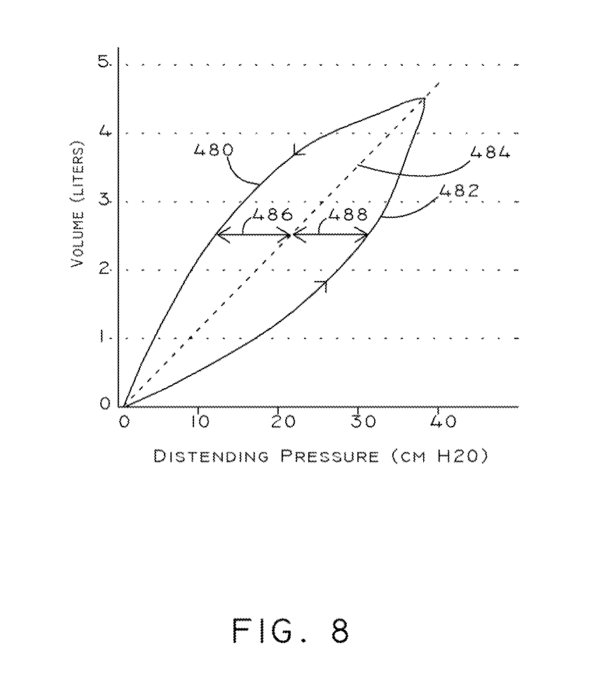

Various techniques exist for measuring static lung compliance. For example, static lung compliance has been measured in the past using the supersyringe method, the multiple-occlusion method, and the constant-flow method. The supersyringe method consists of connecting a supersyringe to the end of an endotracheal tube after allowing the respiratory system to reach relaxation lung volume, and then measuring airway pressure and insufflated volume. The syringe's plunger is moved in regular steps with 2-3 second pauses to allow for quasi-static conditions and then a pressure-volume plot is drawn. Compliance is taken to be the slope of the line in the pressure-volume plot. The pressure-volume plot line will appear somewhat different during inspiration versus expiration (such as illustrated in FIG. 8 described below). However, either can be used, so long as comparison values are measured the same way. In some cases, an overall lung compliance value can be derived by taking the slope of the line connecting the starting and ending points of both the inspiratory and expiratory plot lines (see line 554 of FIG. 8).

The multiple-occlusion method is usually used to get data from awake, cooperating subjects. It involves having the subject first inhale fully, then exhale while periodically interrupting (occluding) the exhalation for 2-3 seconds at different lung volumes and measuring pressure. After each measurement, the subject is generally asked to relax against the occlusion valve. These data points are then plotted on a pressure-volume graph. Again, compliance is taken to be the slope of the line in the pressure-volume plot.

Finally, the constant-flow method involves inflating the lungs at a relatively low constant rate, then deflating at a similar slow rate while observing volume and pressure changes. This method can be used in either awake or anesthetized subjects. The slow flow minimizes pressure contributions of airway resistance, and therefore results in a quasi-static PV curve. This method also includes generating a pressure-volume plot, wherein compliance is the slope of the plot line. Unfortunately, these known techniques generally all require either the combination of an endotracheal tube and a mechanical ventilator (anesthetized subjects) or external flow and pressure monitoring equipment (awake subjects) and an esophageal balloon or other means for measuring pressure in the pleural space. As such, their practical value, particularly in the out-patient context, is somewhat limited.

In contrast to static compliance, the measurement of dynamic compliance is affected by resistive effects of airflow on pressure. However, dynamic compliance is frequently used by clinicians because it is easier to determine and can still be of significant value. Dynamic compliance can be measured during normal tidal breathing simply by observing the volume and pressure changes during a tidal cycle, even in the absence of a constant or zero flow rate. However, specialized equipment is generally still required to capture pressure and volume data. For example, volume is frequently assessed with a pneumotachometer and pressure is frequently assessed with an esophageal pressure balloon and an external pressure transducer. As such, even the measurement of dynamic compliance can be limited to the contexts of clinical visits or in-patient care, when using existing measurement techniques and equipment.

Another relevant pulmonary function parameter is pulmonary resistance. Pulmonary resistance is defined as the driving pressure during airflow divided by the flow rate. The driving pressure can be calculated as the difference between atmospheric pressure and pleural pressure. Reasonably, over a tidal cycle the atmospheric pressure can be treated as constant and therefore changes in driving pressure can be estimated by evaluating the change in pleural pressure (ΔP). The flow rate can be determined directly by a type of flow meter or calculated as the change in volume over the change in time dV/dt. Pulmonary resistance can expressed in units of centimeters of cm H₂O per 1/sec.

Pulmonary resistance can be useful for purposes of monitoring and diagnosing pulmonary and cardiac diseases. However, as with the case of lung compliance, measurements of pulmonary resistance generally require specialized equipment that limit these measurements to clinical visits or in-patient care.

Embodiments described herein can allow the measurement of pulmonary function parameters, such as lung compliance and pulmonary resistance, to be taken without regard to the physical location of the patient or the time of day. Specifically, embodiments of the invention include devices and methods for measuring and/or monitoring pulmonary function parameters using an implanted medical device. The use of an implantable medical device to measure pulmonary function parameters can be advantageous at least because monitoring can be performed as frequently as desired. Aspects of the invention can include methods and implantable medical devices for measuring changes in pressure, methods and implantable medical devices for measuring changes in lung volume and/or flow rate, and methods of calculating pulmonary function parameters. These aspects will now, in turn, be described in greater detail.

### Measurement of Pressure

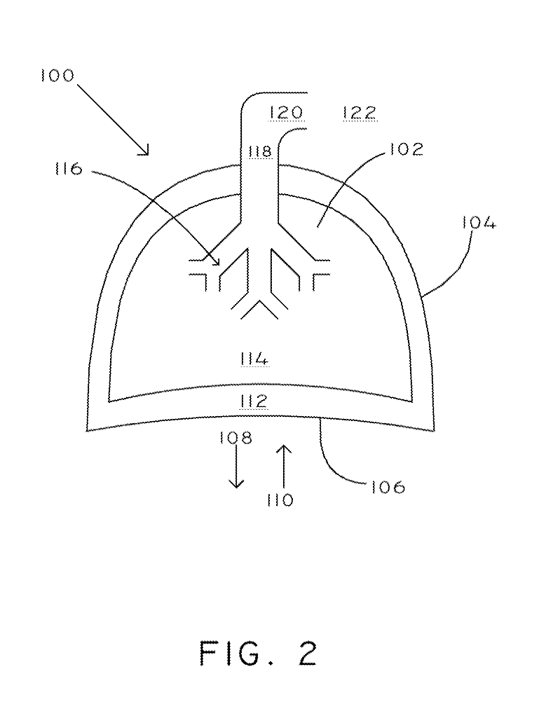

Embodiments of the invention can include methods and devices for measuring changes in pressure with an implanted medical device. Pressures in various parts of the respiratory system are relevant when considering changes that impact the lung. Referring now to FIG. 2, a schematic drawing is shown of the respiratory system 100. The respiratory system includes the lungs 102, the chest wall 104 and the diaphragm 106. The lungs include the bronchi 116 and the alveolar space 114, amongst other things. The pressure within the alveolar space 114 can be referred to as the alveolar pressure. The fluid filled space between the lungs 102 and the chest wall 104 can be referred to as the pleural or intrapleural space 112. The pressure within intrapleural space can be referred to as the pleural pressure. The alveoli 114 are in fluid communication with the trachea 118 and the mouth 120. The mouth, in turn, is in fluid communication with the air outside 122 of the body. It will be appreciated that various other aspects of pulmonary anatomy have been omitted from FIG. 2 for purposes of concise explanation.

The term “driving pressure”, as used herein, refers to a pressure gradient causing air to pass into or out of the lungs 102, from or to the outside 122. The term “distending pressure” as used herein, refers to a pressure gradient that maintains volume of a distensible structure such as the lungs. As such, in some embodiments, distending pressure can include the difference between the alveolar pressure and the pleural pressure. In some embodiments, distending pressure can include the difference between atmospheric pressure and pleural pressure. In some embodiments, driving pressure can be derived from distending pressures at various points of the breathing cycle, or can be measured directly as a difference in pressure along the airways during flow.

Changes in pleural pressure during a respiration cycle can be correlated with changes in distending pressure. The pleural pressure changes during the tidal respiration cycle when the diaphragm 106 in FIG. 2 moves. Specifically, when the diaphragm 106 moves in the direction of arrow 108, the pressure within the intrapleural space is reduced. When the diaphragm 106 moves in the direction of arrow 110, the pressure within the intrapleural space 112 is increased.

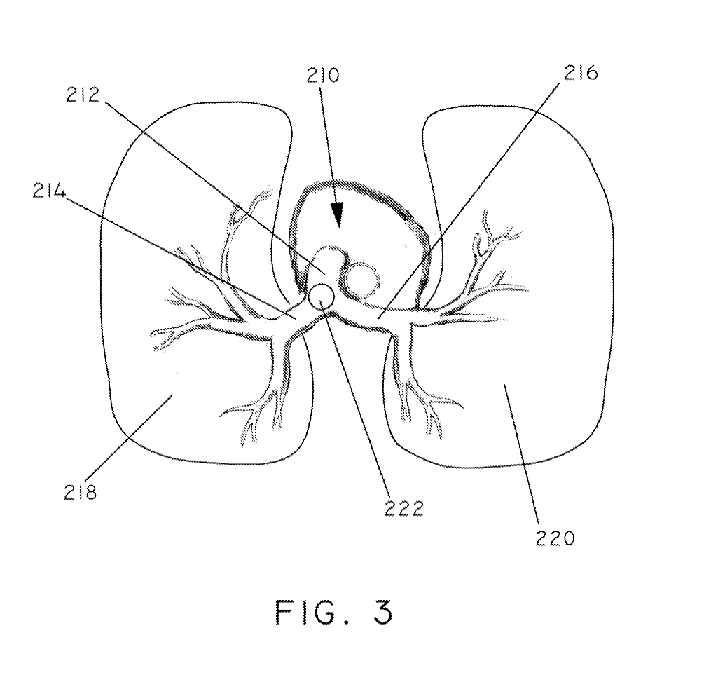

In embodiments of the invention, the pressure within the intrapleural space can be measured or estimated with an implantable medical device. By way of example, in one embodiment, the pressure within the intrapleural space can be measured or estimated using a pressure sensor disposed within the pulmonary artery. While the vasculature commonly referred to as the “pulmonary artery” includes the pulmonary trunk (or main pulmonary artery) and the right and left pulmonary arteries, the term “pulmonary artery” is used in this invention to mean any artery supplying blood to the lungs. FIG. 3 shows a cross-sectional top schematic view of the heart and parts of the pulmonary artery in a human. The pulmonary trunk 212 begins at the base of the right ventricle 210 and extends for approximately 2 inches in length before branching into the left pulmonary artery 214 and right pulmonary artery 216, which deliver deoxygenated blood to the left lung 218 and right lung 220 respectively.

A pressure sensor 222 can be disposed within, or adjacent to, the pulmonary artery in order to generate a signal corresponding to pulmonary artery pressure. The pressure sensor can include any type of sensor, for example, an electrical, mechanical, acoustic, or optical sensor, that generates a signal in response to local pressure or local pressure change. In some embodiments, the pressure sensor can include devices such as those described in U.S. Pat. No. 6,237,398, the contents of which are herein incorporated by reference. The pressure sensor 222 can be configured to transmit pressure data through a conductor or wirelessly.

The pressure sensor 222 can be chronically implanted. The term “chronically implanted” as used herein with respect to a medical device shall refer to those medical devices that are implanted within an organism that are intended to remain implanted long-term, such as for a period of time lasting for weeks, months, or years. Devices described herein can be chronically implanted using standard surgical techniques.

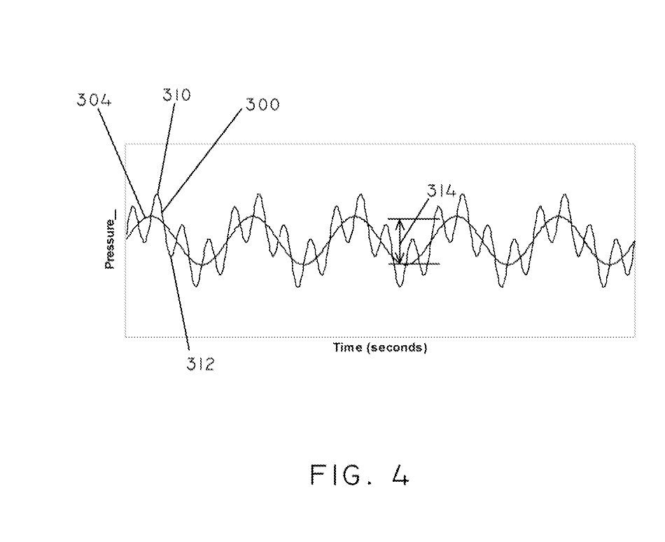

The pressure sensor 222 can produce a signal corresponding to the pulmonary artery pressure. Referring now to FIG. 4, a graph of an idealized pulmonary artery pressure signal 300 is provided for purposes of illustration. It will be appreciated that actual recordings of pulmonary artery pressure within a patient will not necessarily appear identical to the idealized graph of FIG. 4. The pressure signal 300 is a series of peaks 310 and valleys 312, where each peak 310 corresponds to the systolic pressure during the cardiac cycle and each valley 312 corresponds to the diastolic pressure during the cardiac cycle. As can be seen in FIG. 4, the pressure peaks and valleys cyclically rise and fall with time as a result of changes in the intrapleural pressure during inspiration and expiration.

Respiration signal 304 illustrates a roughly sinusoidal respiratory artifact that is superposed on pulmonary artery pressure and reflects changes in pleural pressure during the respiration cycle. Respiration signal 304 can be calculated based on the pulmonary artery pressure signal 300 using various techniques. For example, the respiration signal 304 can be calculated by tracking the fluctuation of the pulmonary artery pressure peaks over time. As another example, the respiration signal 304 can be calculated by tracking the fluctuation of the pulmonary artery pressure valleys over time. The total pressure change between inspiration and expiration 314 can be calculated as the difference in pressure between the peaks and valleys in the respiration signal 304.

In some embodiments, filtering techniques can be used to separate the respiration and cardiac components of pulmonary artery pressure signal 300. For example, a low pass filter with a cutoff frequency of approximately 1-1.2 Hz would substantially pass the respiratory component of the pulmonary artery pressure signal 300, thus creating respiration signal 304, while significantly attenuating the cardiac component. To improve respiratory and cardiac signal separation, the cutoff frequencies of the low pass and high pass filters may be decreased and increased with decreasing and increasing respiratory and/or cardiac rates respectively.

It is believed that measuring changes in intrapleural pressure by measuring the pressure within the pulmonary artery offers at least several advantages. First, a pressure sensor can be disposed within the pulmonary artery using standard minimally invasive percutaneous surgical techniques. Second, because of the anatomical relationship between the pulmonary artery and the intrapleural space, changes in intrapleural pressure can be measured with a pulmonary artery pressure signal accurately.

However, in addition to the pulmonary artery, it will be appreciated that there are also other places within the vasculature of a patient that a pressure sensor can be disposed in, or near, in order to measure intrapleural pressure. By way of example, a pressure sensor can be disposed within an atrium, such as the right atrium, within a ventricle, such as the right ventricle, inside the superior or inferior vena cava, or inside the subclavian vein. In some embodiments, changes in intrapleural pressure can be sensed within the peripheral vasculature.

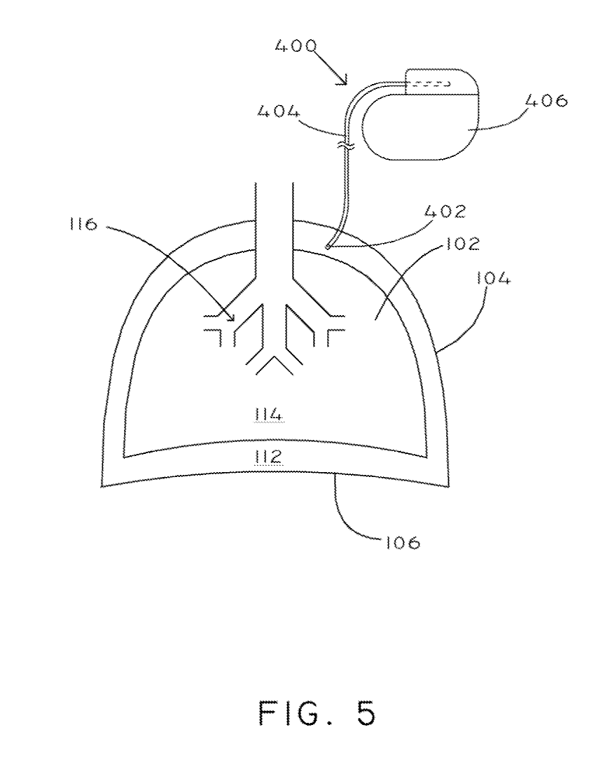

In another embodiment, a pressure sensor can be disposed directly within the intrapleural space itself. The pressure sensor can be tethered to another implantable medical device for wired or wireless communication. Referring now to FIG. 5, an embodiment of an implanted medical device 400 including a pressure sensor 402 in the intrapleural space 112 is schematically shown. The implanted medical device 400 includes a housing 406, the pressure sensor 402, and a sensor lead 404 connecting the housing 406 with the pressure sensor 402. However, in some embodiments, the housing 406 is in wireless communication with the pressure sensor 402. The housing 406 can enclose circuitry such as a processor, memory, a communications module, and the like. In some embodiments, the implanted medical device 400 can be a cardiac rhythm management device. For example, the implanted medical device 400 can be configured to provide electrical stimulation to cardiac tissues for the modulation of cardiac rhythm. Specifically, the implanted medical device 400 can be a pacemaker, a cardiac resynchronization therapy (CRT) device, a remodeling control therapy (RCT) device, a cardioverter/defibrillator, or a pacemaker-cardioverter/defibrillator. One exemplary cardiac rhythm management device is disclosed in commonly assigned U.S. Pat. No. 6,928,325, issued Aug. 9, 2005, the contents of which is herein incorporated by reference.

Beyond pressure sensors, other types of sensors can also be used to generate signals that are indicative of changes in pleural pressure. By way of example, it is believed that changes pleural pressure can result in the diameter of the pulmonary artery changing. As such, the diameter of the pulmonary artery can be measured and correlated with changes in pleural pressure. For example, an accelerometer or pair of magnetometers can be disposed against the wall of the pulmonary artery and changes in the diameter of the pulmonary artery can be sensed by these devices.

It is believed that heart sounds are indicative of pressure changes resulting from respiration. In an embodiment, changes in intrapleural pressure can be estimated by processing a signal reflective of heart sounds. Heart sounds can include the S1, S2, S3, and S4 sounds. By way of example, the S3 heart sound can be measured by a sensor and this signal can be processed and/or filtered in order to estimate changes in intrapleural pressure.

In some embodiments, an implantable transducer can be placed in the esophagus and can be configured to measure intrapleural pressure and transmit a pressure signal to a device either wirelessly or through a lead.

### Measurement of Volume and Flow

Embodiments of the invention can also include methods and devices for measuring the change in volume of the lungs and/or the flow rate of air into or out of the lungs with an implanted medical device. Lung volume changes and flow rates can be measured with an implantable medical device in many different ways. As a specific example, trans-thoracic impedance can be measured in order to assess changes in the volume of the lungs. The blood and body fluids within the thoracic cavity constitute a volume conductor, and the electrical impedance between any two points in the thoracic cavity is dependent upon the volume of blood and/or air between the two points. The impedance can be measured by impressing a constant current field within the cavity and then measuring the potential difference between the two points. By appropriate placement of voltage sensing electrodes, an impedance signal can be produced that corresponds to the movement of air into and out of the lungs as a subject breathes. For example, electrodes can be placed so that impedance vectors primarily capture changes in lung volume.

The resulting impedance signal can then be filtered to derive a volume signal that is proportional to changes in a subject's lung volume due to breathing. An exemplary technique for measuring transthoracic impedance with an implantable medical device is described in commonly assigned U.S. Pat. No. 6,868,346, the contents of which are herein incorporated by reference.

In another embodiment, dimensional changes of the lung can be used to derive changes in lung volume. Dimensional changes of the lung can be assessed in various ways. For example, dimensional changes of the lung can be detected using an accelerometer disposed adjacent to the lung wall or a pair of magnetometers affixed to different points on the lung wall or thoracic cage.

Because of impedance effects, bio-electric properties within the chest cavity can fluctuate based on the volume of the lungs. As such, signals reflecting bio-electric properties, such as electrogram signals, can be filtered and/or processed in order to derive lung volume change during breathing. In some embodiments, lung volume change during breathing is assessed by analyzing electrogram signals. Cardiac intervals, such as P-R intervals or R-R intervals, can also be modulated by respiration. As such, in some embodiments, data regarding cardiac intervals is evaluated in order to derive lung volume change during breathing.

The lungs can exert pressure on the heart as the volume of air within the lungs increases. As a result, stroke volume can be reduced in proportion to the volume of air in the lungs. In some embodiments, stroke volume can be measured with a sensor, such as a flow sensor, and then stroke volume data can be filtered and/or processed in order to derive lung volume change during breathing. In some embodiments, a sensor for detecting lung volume change during breathing can be chronically implanted.

In another embodiment, an implantable flow sensor can be disposed within a main airway segment such as within the trachea, larynx or mouth. The flow sensor can be configured to generate a signal indicative of the air flow into and out of the lungs. This signal can be received by devices as described herein and used to calculate various pulmonary function parameters.

It is believed that the signal from an implanted accelerometer for detecting heart sounds will shift based on volume changes associated with the tidal respiration cycle. Specifically, it is believed that the S1 heart sound is modulated by changes in lung volume. In some embodiments, the signal from an accelerometer can be filtered and/or processed in order derive lung volume change during breathing. For example, a signal representing the S1 heart sound can be filtered and/or processed in order to derive lung volume change during breathing.

In some embodiments, lung volume change during breathing can be assessed with a separate piece of equipment outside of the body and then volume data can be wirelessly transmitted to an implanted medical device within the body for calculation of lung compliance. For example, lung volume change during breathing can be derived through the use of a spirometer or pneumotachometer, or positive pressure device (such as CPAP) and then a signal representing this data can be wirelessly transmitted to an implanted medical device within the body for further processing.

It is believed that total body electrical impedance and thoracic electrical impedance is correlated with lung volume. As such, total body impedance or thoracic cage impedance can be assessed through the use of electrodes outside of the body, and then total body impedance can be used to derive lung volume.

A further approach using equipment outside of the body is respiratory inductance plethysmography and variations thereof. In this technique, a subject wears two inductance bands, for example one around the ribcage and the other around the abdomen. Lung volume change during breathing can then be derived from changes in the inductance of the coils. Closely related to this approach is the assessment of dimensional changes of the lung using impedance bands placed around the external chest of the subject.

In another embodiment, changes in lung volume change during breathing can be assessed using a nasal cannula flow sensor. In this approach, a nasal cannula flow sensor can be clipped to the nose of a subject can configured to measure airflow through the nasal passage.

In embodiments where an external device is used to measure lung volume change during breathing, a signal related to lung volume change during breathing can be transmitted back to an implanted device for further operations including calculation of lung compliance and/or pulmonary resistance. The signal can be transmitted in various ways including radiofrequency (RF) transmission, inductance, acoustically, and the like. In some embodiments, the signal can be transmitted to an implanted device through an advanced patient management system. Exemplary advanced patient management systems are described in greater detail below.

It will be appreciated that techniques described above for measuring lung volume change during breathing can be used to measure relative lung volume and, in some cases, can also be used to measure absolute lung volume. For example, in the context of measuring impedance, impedance values can be calibrated such that an approximation of absolute lung volume can be derived. In one approach for calibration, after an impedance sensor is implanted within a patient, the patient can be directed to breathe into a spirometer, or similar device, while the transthoracic impedance is measured. The correlation function between the transthoracic impedance signal and lung volume can then be derived, stored, and later applied to approximate an absolute measurement of lung volume.

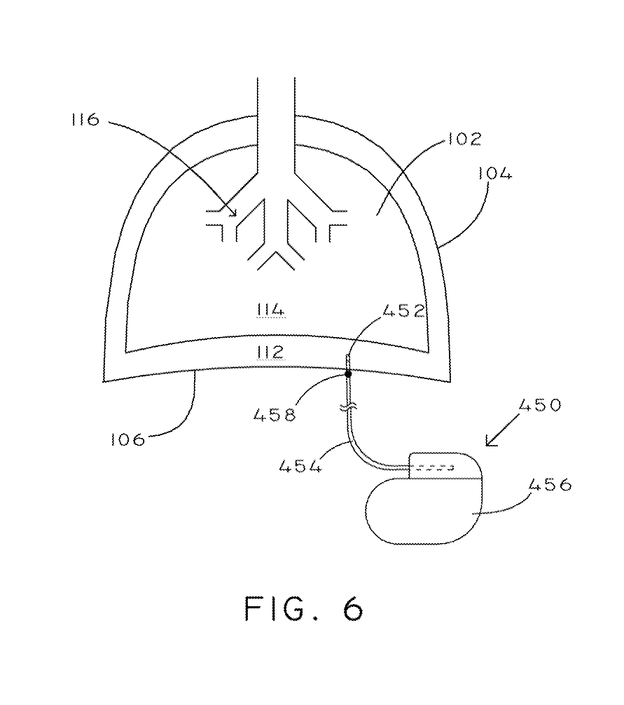

Sensors for measuring volume and flow can be implemented in various configurations. For example, in some embodiments, a sensor for measuring lung volume change during breathing or flow rate and a sensor for measuring intrapleural pressure can be coupled to a single lead. Referring now to FIG. 6, an embodiment of an implanted medical device 450 is schematically shown with both a pressure sensor and a volume sensor coupled to a single lead. A pressure sensor 452 is disposed in the intrapleural space 112. A volume sensor 458 is disposed just outside the diaphragm 106. The volume sensor 458 can be an accelerometer and can generate a signal in response to movements of the diaphragm. Both the pressure sensor 452 and the volume sensor 458 are coupled to a lead 454. The lead can include a conductor and can be configured to convey pressure and volume signals from the sensors to implanted medical device housing 456. The housing 456 can enclose circuitry such as a processor, memory, a communications module, and the like, for performing various operations with pressure and volume signals.

### Calculation of Pulmonary Function Parameters

Embodiments of the invention can include methods of calculating pulmonary function parameters based on measurements of pleural pressure and lung volume change during breathing or flow rate. Exemplary pulmonary function parameters can include lung compliance, pulmonary resistance, pressure-volume loop area, press-volume loop centroid and the like.

Embodiments of the invention can specifically include methods of calculating lung compliance based on measurements of pleural pressure and lung volume change during breathing. As described above, lung compliance (C_{L}) is defined as the change in lung volume (ΔV) per unit pressure change (ΔP). Signals corresponding to pressure changes (ΔP) and volume changes (ΔV) can be derived through many different techniques, such as those described above. These signals can then be processed in order to calculate lung compliance. Specifically, the change in volume can be divided by the change in pressure in order to calculate lung compliance.

Embodiments of the invention can also include methods of calculating pulmonary resistance based on measurements of pleural pressure and volume and/or flow rate. As described above, pulmonary resistance is defined as the driving pressure divided by the flow rate. The driving pressure can be calculated in various ways, such the as the atmospheric pressure minus pleural pressure. The flow rate can be determined directly or indirectly through various techniques such as those described above. In general, any technique for measuring lung volume change during breathing can be applied in the context of measuring flow rate by simply adding a measure of time (e.g., flow rate equal volume change divided by time).

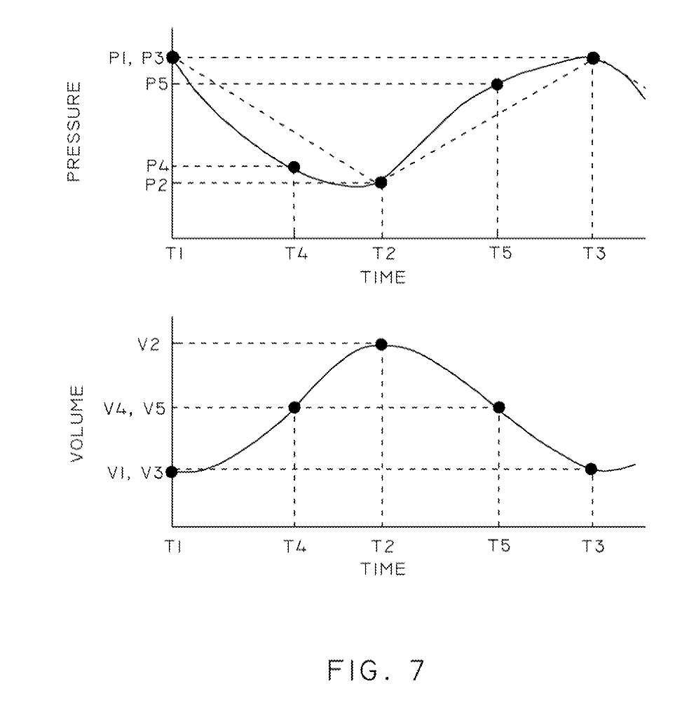

After obtaining pressure and volume data, pulmonary resistance can be calculated in various ways. One specific technique is described herein with reference to FIG. 7, which shows an approximation of both an idealized pressure plot and an idealized volume plot. It will be appreciated that actual pressure and volume plots from patients can be somewhat different from that shown in FIG. 7. Using the pressure and volume data from these plots, pulmonary resistance can be estimated according to the following formulas:

$$Inspiratory Pulmonary Resistance = \frac{\frac{P 1 + P 2}{2} - P 4}{\frac{V 2 - V 1}{T 2 - T 1}}$$

$$Expiratory Pulmonary Resistance = \frac{P 5 - \frac{P 3 + P 2}{2}}{\frac{V 2 - V 3}{T 3 - T 2}}$$

Wherein T1, T2, and T3 are identified by peaks and valleys of the volume signal. V4 and V5 are volumes at the mid-point of the breathing cycle, on the inspiratory and expiratory limbs, respectively. T4 is the time point of mid-volume during inspiration (time point corresponding to V4). T5 is the time point of mid-volume during expiration (time point corresponding to V5).

It will be appreciated that there are also many other methods and formulas that can be used to calculate or estimate pulmonary resistance using pressure and flow rate data beyond those illustrated in the foregoing specific example.

Using the pressure and volume data from the plots of FIG. 7, dynamic compliance can be estimated according to the following formula:

$$Dynamic Compliance = \frac{V 2 - V 1}{P 1 - P 2}$$

Referring now to FIG. 8, a hypothetical pressure-volume plot for a healthy lung is shown illustrating both expiration and inspiration. Plot line 480 represents the pressures and volumes measured during expiration. Plot line 482 represents the pressures and volumes measured during inspiration. Plot lines 480 and 482 are not identical because of various effects including hysteresis and driving pressure of flow. In this embodiment, the slope of plot line 484 can be used as an overall value for dynamic lung compliance.

The area bounded by plot lines 480 and 482 can be referred to as the loop area. Loop area can be easily calculated once plot lines 480 and 482 are determined. The pressure-volume loop centroid is the point whose coordinates are the averages of the set of points making up both the expiratory plot line 482 and the inspiratory plot line 482. The loop centroid can also be easily calculated once plot lines 480 and 482 are determined. Distance 486 represents the driving pressure to overcome expiratory pulmonary resistance. Expiratory pulmonary resistance can be measured in cm H₂O per 1/sec. Distance 488 represents the driving pressure to overcome inspiratory pulmonary resistance. Inspiratory pulmonary resistance can also be measured in cm H₂O per 1/sec. Overall pulmonary resistance can be calculated as the average of expiratory pulmonary resistance and inspiratory pulmonary resistance.

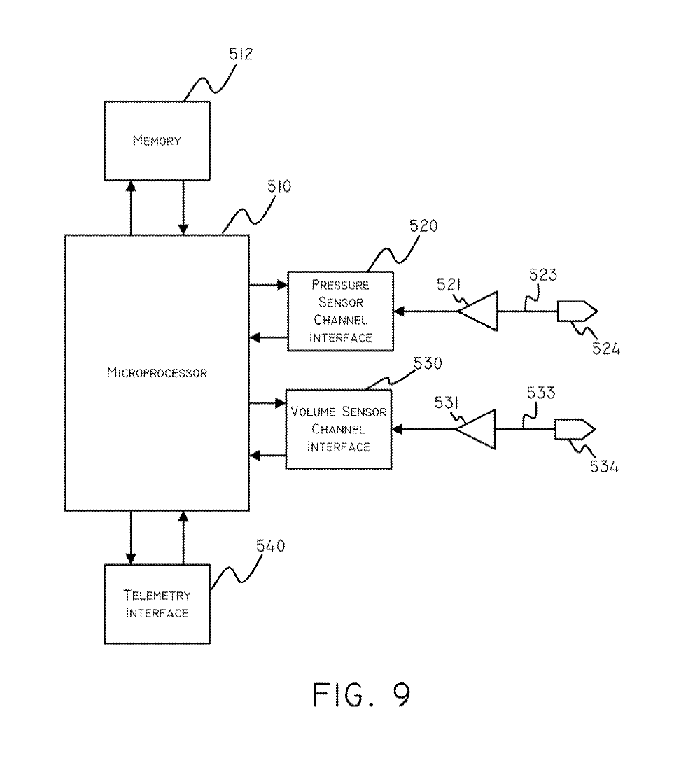

A particular implementation of an implantable medical device that can be configured to determine pulmonary function parameters is shown schematically in FIG. 9. A microprocessor 510 serves as the controller in this embodiment and communicates with memory 512 via a bidirectional data bus. The memory 512 typically comprises ROM (read-only memory) or RAM (random access memory) for program storage and RAM for data storage. The implantable medical device has a pressure sensor channel comprising pressure sensor 524, lead 523, sensing amplifier 521, and a pressure sensor channel interface 520 which can communicate bidirectionally with a port of microprocessor 510. In this embodiment, the device also has a volume sensor channel comprising volume sensor 534, lead 533, sensing amplifier 531, and a volume sensor channel interface 530 which can communicate bidirectionally with a port of microprocessor 510. It will be appreciated that in some embodiments the sensors and/or amplifiers can be in wireless communication to the channel interfaces or the microprocessor.

The channel interfaces 520 and 530 can include analog-to-digital converters for digitizing signal inputs from the sensors and registers which can be written to by the microprocessor in order to initiate sensing, change sensing parameters, adjust the gain and threshold values for the sensing amplifiers, and the like. A telemetry interface 540 is also provided for communicating with an external device, such as an external programmer or an advanced patient management system. The system can also include other components not shown such as a power source, and the like.

The microprocessor 510, in conjunction with other components described herein, can be configured to execute methods described herein, including calculating pulmonary function parameter values. In addition, the microprocessor 510 and related components can initiate activation of the pressure sensor through the pressure sensor channel interface and can initiate activation of the volume sensor through the volume sensor channel interface. In some embodiments, the pressure sensor and the volume sensor are activated (turned on) and generating pressure and volume signals continuously.

However, in the context of chronically implanted medical devices, it can be desirable to operate the system so as to conserve battery life. As such, in some embodiments, the pressure sensor and the volume sensor are operated intermittently. For example, the pressure sensor and the volume sensor can be activated for a period of time long enough to gather data sufficient to calculate pulmonary function parameters and then shut off. In some embodiments, the pressure sensor and the volume sensor are activated (turned on) at least once per day.

In some embodiments, the mode of operation of the device can change depending on recent values of pulmonary function parameters. Specifically, in some embodiments the frequency with which pulmonary function parameter measurements are taken can change depending on what the current pulmonary function parameter measurement illustrates. For example, it can be desirable to identify an adverse pulmonary function trend as early as possible, and adverse trends can generally be more accurately identified with the benefit of a larger number of data points. As such, in some embodiments, when a pulmonary function parameter value is measured by the system that deviates from a baseline value for that patient by at least a threshold amount, the system changes the frequency of measuring the pulmonary function parameter so as to be able to capture additional data points.

By way of illustration, in some embodiments, the device may be configured to only measure a pulmonary function parameter once per day. However, if measured pulmonary function parameter differs from a baseline value or reference value for a particular patient by at least a threshold amount, then the device changes its mode of operation to measure the pulmonary function parameter more frequently, such as once per hour. In some embodiments, if a measured pulmonary function parameter differs from a baseline value by at least a threshold amount for a certain number of discrete measurements (samples), then the device can change its mode of operation. For example, if the measured pulmonary function parameter exceeds a threshold amount 5 times out of 10, then the device can change its mode of operation. If after a period of time of measuring at the higher frequency no adverse trend is identified, then the system can resume measuring the pulmonary function parameter at the lower frequency, such as once per day in this illustration.

In some embodiments, the threshold amount is a based off of a statistical measure of prior values. For example, the threshold amount could be set equal to a certain multiple of the standard deviation of past measurements. In one embodiment, the threshold amount can be the standard deviation of measurements taken over the last day, or the last week. In another embodiment, the threshold amount can be twice the standard deviation of measurements taken over the last day, or the last week.

It is known that various factors including posture, physical activity, time of day, etc. can impact measurements of pulmonary function parameters such as lung compliance and pulmonary resistance. As such, when identifying trends it can be desirable to ensure that comparisons are made between discrete measurements taken under similar circumstances. As a specific example it is known that the posture of the patient, can impact the measured pulmonary function parameter value. As such, in some embodiments, a posture sensor is used in order to evaluate when to activate the pressure and volume sensors. A signal from the posture sensor can be used to generate a triggering signal that causes the device to initiate pressure and volume measurements. In some embodiments, pulmonary function parameter measurements are only taken when the patient is in the same posture as during previous pulmonary function parameter measurements. For example, in some embodiments, pulmonary function parameter measurements are only taken when the patient is standing. In other embodiments, pulmonary function parameter measurements are only taken when the patient is lying supine.

In some embodiments, a physical activity sensor, such as an accelerometer, is used in order to evaluate when to activate the pressure and volume sensors. A signal from the activity sensor can be used to generate a triggering signal that causes the device to initiate pressure and volume measurements. In some embodiments, pulmonary function parameter measurements are only taken when the patient is at the same state of physical activity as during previous pulmonary function parameter measurements. For example, in some embodiments, pulmonary function parameter measurements are only taken when the patient is physically inactive.

Some patients can exhibit a circadian rhythm with respect to pulmonary function parameters. This can be attributed to circadian rhythms with respect to fluid in their lungs. Because of these circadian rhythms, it can be difficult to discern a subtle pulmonary function trend in some patients, if data from a given time of the day is being compared against data from a different time of the day. Accordingly, the implantable medical device can include a clock. In some embodiments, a triggering signal can be generated at a particular time of day. For example, in some embodiments the triggering signal can be generated during the day time. In some embodiments, the triggering signal can be generated during the night time, such as between midnight and 6:00 AM. In some embodiments, the pressure and volume measurements are taken at the same time, or during the same window of time, as when previously recorded pressure and volume measurements were taken. In some embodiments, the window of time is less than or equal to eight hours. In some embodiments, the window of time is less than or equal to six hours. In some embodiments, the window of time is less than or equal to four hours.

In some embodiments, the triggering signal can be generated in response to a combination of condition indicators. For example, the triggering signal can be generated in response to a combination of posture, physical activity, time of day, etc.

In some embodiments, data can be gathered without regard to the current measurement conditions (posture, physical activity, time of day, etc.), however the data are only compared with data taken under similar conditions when evaluating trends. For example, data can be gathered regardless of the posture of the patient, but for purposes of evaluating trends data taken when the patient is in a supine position, for example, will only be compared with other data taken when the patient is in a supine position. In this manner, multiple trends can be generated for the pulmonary function parameters corresponding to different measurement conditions. For example, a supine trend and a standing trend can be generated. As another example, a daytime trend and a nighttime trend can be generated.

### Active and Passive Measurement

In some embodiments, pulmonary function parameters can be assessed passively, where a patient with an implanted device simply breathes normally and the pulmonary function parameter is calculated as desired (such as intermittently or continuously) without any specific action of the patient.

However, in other embodiments, pulmonary function parameters can be assessed actively, where the patient is triggered to perform a respiratory maneuver, such as exhale or inhale, and then the pulmonary function parameter is measured during this prompted maneuver. In some embodiments, a signal can be given to a patient indicating that they should alter their breathing, such as inhaling as deeply as possible or exhaling as deeply as possible. The signal can be a sound, such as a beep, a kinetic signal, such as a buzzing sensation, or the like.

Some patients may have poor control over voluntary breathing functions. In addition, some patients may be unable to follow directions given to them, such as unconscious patients. Accordingly, in some embodiments, a pulmonary function parameter can be assessed actively by stimulating contraction of the diaphragm. For example, an electrical stimulation pulse can be administered to the phrenic nerve in order to stimulate contraction of the diaphragm. The phrenic nerve acts as the motor nerve of the diaphragm. It runs through the thorax, along the heart, and then to the diaphragm. Alternately, the diaphragm itself can be stimulated directly. Contraction of the diaphragm causes it to move in the direction of arrow 108 (shown in FIG. 2), lowering pleural pressure and triggering inspiration. Generally, the diaphragm has a higher stimulation threshold than chambers of the heart. Exemplary methods and systems for stimulating the phrenic nerve to cause contraction of the diaphragm can be found in commonly assigned U.S. Pat. No. 6,415,183, the contents of which are herein incorporated by reference.

In some embodiments, the implanted device is configured to induce contraction of the diaphragm at regular intervals, such as once per day. In other embodiments, the implanted device is configured to induce contraction of the diaphragm when it receives an initiation signal from outside the body, such as from a programmer device or advanced patient management device.

### Diagnosis, Management, and Monitoring of Pulmonary or Cardiac Diseases

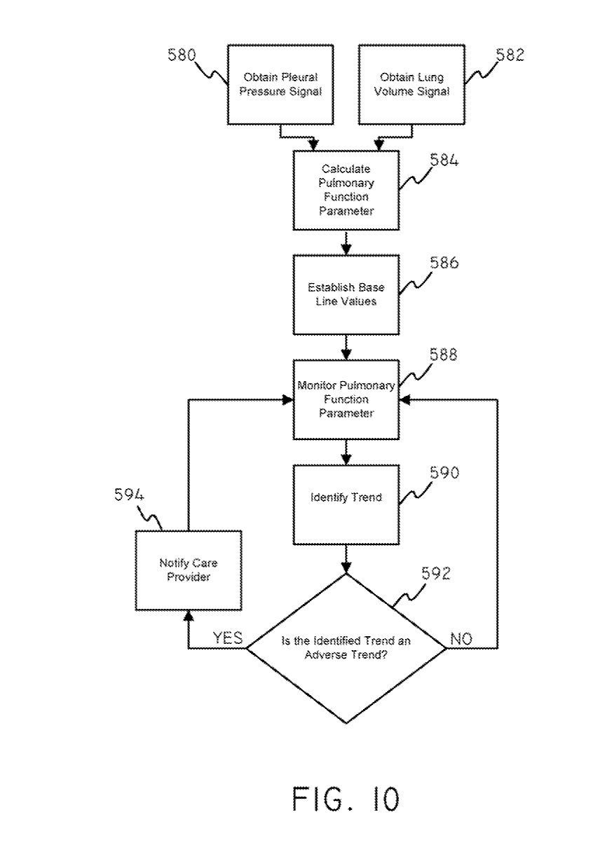

Pulmonary function parameter trends can be a valuable tool for clinicians when monitoring or evaluating various pulmonary or cardiac diseases or conditions. For example, adverse pulmonary function trends may indicate that medical intervention is required. Systems and methods of the invention can include the ability to identify adverse pulmonary function parameter trends and then appropriately act upon the same. For example, referring now to FIG. 10, a flowchart is shown of a method for monitoring a pulmonary function parameter trend. The method includes obtaining a pleural pressure signal 580 and obtaining a lung volume signal 582. The method also includes calculating a pulmonary function parameter 584 based on the pleural pressure signal 580 and the lung volume signal 582. The method also includes establishing a base line value for the pulmonary function parameter 586. Establishing base line pulmonary function parameter values 586 for a particular patient allows the system to accommodate differences between individuals with regard to their normal pulmonary function. The base line value may include values taken from a single prior period, or it may include a set of data taken over a significant period of time.

The method also includes monitoring pulmonary function parameters. This is done by periodically calculating the value for a pulmonary function parameter in real time and comparing it with a reference value, such as the base line value. Next, the method can include identifying the pulmonary function parameter trend 590. The pulmonary function parameter trend may indicate that the pulmonary function parameter value is increasing over time, decreasing over time, or holding steady. The method also includes evaluating whether or not the identified pulmonary function parameter trend is adverse 592. An adverse trend can include one or more pulmonary function parameter measurements that deviate from the baseline value by at least a threshold amount. For example, in some embodiments, an adverse trend can include one or more consecutive measurements that deviate from the baseline pulmonary function parameter value by at least one standard deviation, or some multiple of a standard deviation. In some embodiments, an adverse trend can include two or more consecutive measurements that deviate from the baseline pulmonary function parameter value by at least one standard deviation. In some embodiments, the criteria for identifying a trend as adverse can be configured by a care provider.

If an adverse trend is identified, then a care provider can be notified 594. This can include sending an alert or message to a care provider through an external unit, such as an advanced patient management system. An exemplary advanced patient management system is the LATITUDE® patient management system, commercially available from Boston Scientific Corporation, Natick, Mass. Aspects of an exemplary advanced patient management system are described in U.S. Pat. No. 6,978,182, the contents of which are herein incorporated by reference. The notification can also include aural alerts such as beeps and the like. However, if an adverse trend is not identified, then the method can include going back to the step of monitoring the pulmonary function parameter 588 and continuing on. In some embodiments, if an adverse trend is identified then appropriate therapy can be administered. Appropriate therapy can include administration of therapeutic agents and the like.

Changes in pulmonary function parameters can be used in order to aid in the diagnosis of various disease states. For example, increasing magnitude of expiratory pulmonary resistance relative to inspiratory pulmonary resistance can be indicative of one or more forms of chronic obstructive pulmonary disease (COPD). As such, in some embodiments, values for inspiratory pulmonary resistance and expiratory pulmonary resistance (referred to collectively as “pulmonary resistance”) are calculated and monitored over time.

In addition, in some embodiments, the combination of changes in lung compliance and pulmonary resistance (both inspiratory and expiratory) can be used in order to predict whether observed changes are more likely to stem from a cardiac condition (cardiogenic changes) or a pulmonary condition (pulmogenic). For example, Table 1 below illustrates expected effects of various conditions on lung compliance and pulmonary resistance (inspiratory and expiratory) in various cardiac and pulmonary conditions.

TABLE 1

| Disease | | | Inspiratory | Expiratory |
|---|---|---|---|---|
| Type | Disease | Compliance | Resistance | Resistance |
| Cardiac | Chronic | Decreased, | Normal | Normal |
| | Heart | but Stable | | |
| | Failure | | | |
| | Acute | Decreased, | Normal or | Normal or |
| | Edema | Rapid Onset | Increased | Increased |
| Pulmonary | Asthma | Normal or | Increased, | Increased, |
| | (acute) | Decreased, | Rapid | Rapid |
| | | Rapid | Onset in | Onset in |
| | | Decrease in | Acute | Acute |
| | | Acute | Attacks | Attacks |
| | | Attack | | |
| | Bronchitis | Normal or | Increased, | Increased, |
| | | Decreased, | Rapid | Rapid |
| | | Rapid | Onset in | Onset in |
| | | Decrease in | Acute | Acute |
| | | Acute | Attacks | Attacks |
| | | attack | | |
| | Emphysema | Increased, | Normal | Increased |
| | | Slowly | | |
| | | Increasing | | |
| | Fibrosis | Decreased, | Normal or | Normal or |
| | | but Stable | Decreased | Decreased |
| | | or Slowly | | |
| | | Decreasing | | |

It will be appreciated that pulmonary function parameter trends can be useful in identifying and monitoring many different conditions and diseases. Referring now to FIG. 11, a hypothetical graph of lung compliance over time illustrating changes common in pulmonary edema is shown. During a first time period 602, the compliance values exhibit an amount of variation around a normal value. This amount of variation would be consistent with signal and measurement noise. However, during a second time period 604, the lung compliance rapidly drops off over a period of days signaling dramatically worsening pulmonary edema. In some cases, this may be indicative of rapidly progressing heart failure decompensation. In any case, this would justify further evaluation and/or treatment.

As such, in some embodiments, when the device identifies a pulmonary function parameter trend indicative of pulmonary edema, the device creates a signal or alert so that further action can be taken by a care provider. The signal or alert can be relayed via telemetry to an external device and then delivered to a care provider for further action. In some embodiments, the pulmonary function parameter data, and the signal or alert can be transmitted to an advanced patient management system, and then delivered to a care provider.

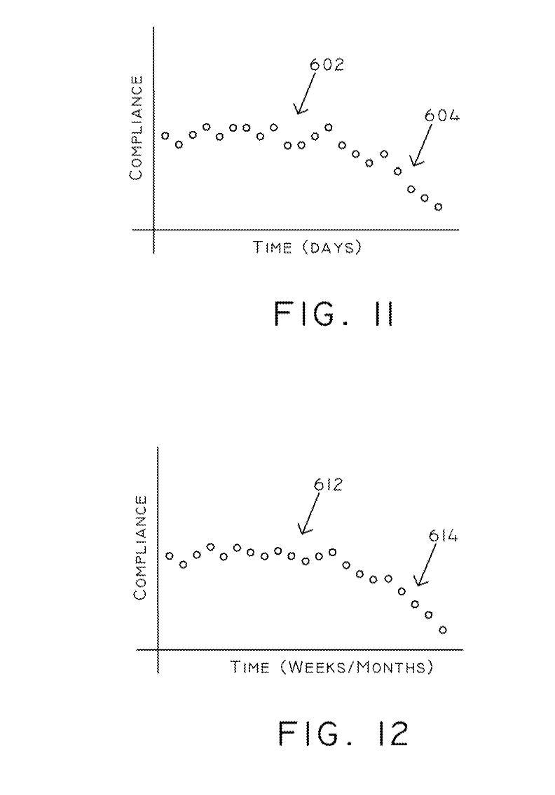

FIG. 12 is a hypothetical graph of lung compliance over time illustrating changes common in pulmonary fibrosis. During a first time period 612, the lung compliance values exhibit an amount of variation around a normal value. This amount of variation would be consistent with signal and measurement noise. However, during a second time period 614, the lung compliance steadily drops off over a period of weeks or months signaling gradually worsening pulmonary fibrosis. This type of adverse trend in lung compliance can justify further evaluation and/or treatment. In some embodiments, the pulmonary function parameter data is stored and then transmitted to an external unit for presentation to a care provider. However, in other embodiments, the device creates a signal or alert so that further action can be taken by a care provider. The signal or alert can be relayed via telemetry to an external device, such as an advanced patient management system, and then delivered to a care provider for further action.

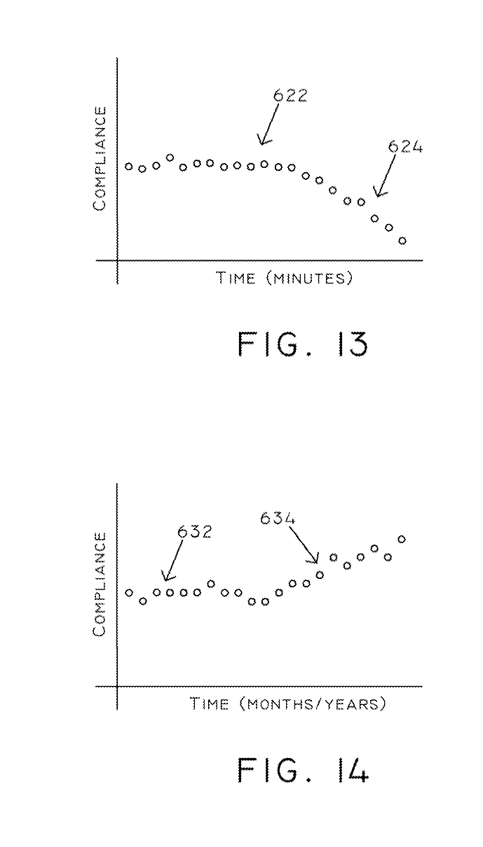

FIG. 13 is a hypothetical graph of lung compliance or pulmonary resistance over time illustrating changes common in an asthma attack. During a first time period 622, the lung compliance or pulmonary resistance values exhibit an amount of variation around a normal value. This amount of variation would be consistent with signal and measurement noise. However, during a second time period 624, the lung compliance suddenly drops off or pulmonary resistance suddenly increases over a short period of time, such as 30 to 60 minutes, signaling a possible asthma attack. In some embodiments, this data is collected and then presented to a care provider the next time the device is interrogated. This type of data can be valuable when, for example, a care provider is trying to evaluate the effectiveness of a patient's current asthma drug regimen. In other embodiments, a pulmonary function parameter trend indicating an asthma attack can be used to initiate appropriate therapy. For example, this data can be used in conjunction with a drug delivery device to administer an active agent that can ameliorate the asthma attack.

FIG. 14 is a hypothetical graph of lung compliance over time illustrating changes common in emphysema. During a first time period 632, the lung compliance values exhibit an amount of variation around a normal value. This amount of variation would be consistent with signal and measurement noise. However, during a second time period 634, the lung compliance gradually increases over a period of years signaling gradually worsening emphysema. This type of adverse trend in lung compliance can be extremely useful to a care provider for purposes of making treatment decisions and evaluating a particular patient's prognosis. In some embodiments, the pulmonary function parameter data is stored and then transmitted to an external unit for presentation to a care provider.

While lung compliance trends over time have been illustrated herein with respect to pulmonary edema, pulmonary fibrosis, asthma, and emphysema, it will be appreciated that lung compliance trends can be used as an aid in diagnosing and monitoring many other types of pulmonary diseases and conditions as well. It will also be appreciated that these disease states will also impact other pulmonary function parameters, such as pulmonary resistance. Accordingly, pulmonary function parameters other than lung compliance can also be monitored in order to aid in diagnosing and monitoring pulmonary diseases.

It will be appreciated that pulmonary function parameters can also be a valuable tool in monitoring or evaluating diseases other than just pulmonary diseases. For example, heart failure decompensation is known to result in pulmonary edema, which affects pulmonary function parameters. As a specific example, rapidly declining lung compliance in a heart failure patient may be an indication that immediate medical intervention is required. Embodiments of the invention can be configured to identify this adverse trend and alert care providers. In some embodiments, a pulmonary function parameter trend indicating rapidly declining pulmonary function in a heart failure patient can be used to initiate appropriate therapy. For example, this data can be used in conjunction with a drug delivery device to administer an active agent, such as a diuretic, that can counter the rapid increase in pulmonary edema.

Embodiments of methods herein can also include methods of titrating or adjusting drug therapy in a closed loop system. Various medications can impact pulmonary function parameters and therefore the effects of various medications can be monitored and changes in the drug regimen can be initiated based on changes in a pulmonary function parameter. For example, diuretic drug therapy can affect lung compliance. Rapidly rising or falling lung compliance may indicate that either too much or too little diuretic medication is being administered. As such, in some embodiments, an increase of the dosage of a diuretic medication can be initiated in response to rapidly falling lung compliance. In other embodiments, a decrease of the dosage of a diuretic medication can be initiated in response to a rapidly rising lung compliance. It will be appreciated that the dosage of other types of medications, beyond diuretics, can similarly be titrated according to methods described herein. In addition, values of other pulmonary function parameters, such as pulmonary resistance, can similarly be used in methods of titrating or adjusting drug therapy in a closed loop system.

Embodiments of the invention can also include titration of agents for treating the class of diseases referred to as chronic obstructive pulmonary disease (COPD). Agents used to treat COPD can include bronchodilators (such as albuterol, levalbuterol, pirbuterol acetate, terbutaline sulfate, salmeterol xinafoate, formoterol), theophylline agents (such as theophylline, aminophylline), cholinergic blockers (ipratropium bromide), leukotriene modifiers (such as montelukast sodium), anti-inflammatory medications (such as steroids). As with many therapeutic agents, dosages of these agents that are too high may cause side effects while dosages that are too low may not provide the desired therapeutic effect. As such, there is a need to adjust the dosage of these agents as taken by COPD patients.

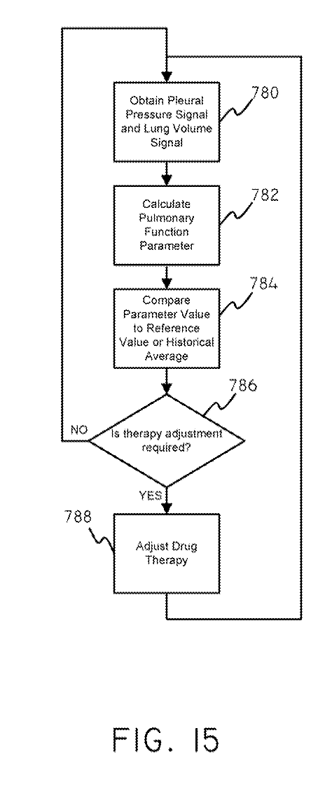

Referring now to FIG. 15, a flow chart is shown illustrating a method of titrating drug therapy. The method includes obtaining a pleural pressure signal and obtaining a lung volume signal 780. The method also includes calculating 782 a pulmonary function parameter based on the pleural pressure signal and the lung volume signal. The method also includes comparing 784 pulmonary function parameter values with normal or historical pulmonary function parameter values. In some embodiments, the pulmonary function parameter values can be real-time or near real-time values. The normal value for a pulmonary function parameter can be values that are normal for a specific patient or values that are normal as calculated for a patient population. Then, the question of whether or not therapy adjustment is required can be assessed 786. This will depend on various parameters, such as the specific disease that the patient suffers from and the particular drug being administered. If the answer is no, then the steps can be repeated, starting with obtaining a pleural pressure signal and obtaining a lung volume signal 780. However, if the answer is yes, then the drug therapy can be adjusted (such as increasing or decreasing the dosage) before repeating the steps from the beginning.

It will be appreciated that measurement of pulmonary function parameters can be of value to clinicians under a variety of circumstances. As such, in some circumstances, care providers may desire to review real-time data regarding pulmonary function parameters. In some embodiments, implantable devices herein can be configured to be activated by an initiation signal and to produce one or more signals reflective of pulmonary function parameters that can be received by an external device and then displayed for a care provider in real time. For example, a care provider can send an initiation signal to an implantable device through an external programmer device or through an advanced patient management system causing the device to initiate real-time monitoring of one or more pulmonary function parameters. This data can then be transmitted back to an external unit such as a programmer or an advanced patient management system and the data can be displayed for the care provider in real time through a display screen. In some embodiments, the care provider can trigger the initiation signal remotely such as through a web interface and receive the real-time data remotely through the same interface.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A method of determining a pulmonary function parameter of a subject, the method comprising
obtaining a first signal indicative of lung volume change during breathing from a first sensor;
obtaining a second signal indicative of lung distending pressure from a second sensor, wherein at least one of the first and second sensors are chronically implanted; and
calculating the pulmonary function parameter based on the first signal and the second signal.

2. The method of claim 1, the pulmonary function parameter selected from the group consisting of lung compliance, pulmonary resistance, pressure-volume loop area, and pressure-volume loop centroid.

3. The method of claim 1, further comprising prompting the subject to perform a respiratory maneuver.

4. The method of claim 1, further comprising electrically stimulating contraction of the diaphragm.

5. The method of claim 1, further comprising electrically stimulating the phrenic nerve.

6. The method of claim 1, wherein both the first sensor and the second sensor are chronically implanted.

7. The method of claim 1, wherein obtaining a first signal indicative of lung volume change during breathing from a first sensor comprises obtaining an impedance signal from an impedance sensor.

8. The method of claim 1, wherein obtaining a second signal indicative of distending pressure from a second sensor comprises obtaining a pulmonary artery pressure signal from a pressure sensor.

9. The method of claim 1, wherein obtaining a second signal indicative of distending pressure from a second sensor comprises obtaining a pleural pressure signal.

10. The method of claim 1, further comprising transmitting the first signal and second signal to a processing unit.

11. The method of claim 1, wherein obtaining the first signal and obtaining the second signal are performed during normal tidal breathing of the patient.

12. The method of claim 1, wherein obtaining the first signal and obtaining the second signal are only performed in response to a triggering signal.

13. The method of claim 12, the triggering signal related to posture of the subject, wherein the triggering signal indicates that the subject is in a supine position.

14. The method of claim 12, the triggering signal related to the time of day.

15. The method of claim 12, the triggering signal related to the activity level of the subject, wherein the triggering signal indicates that the subject is at a particular level of activity.

16. A method of monitoring pulmonary or cardiac disease status, the method comprising:
obtaining a first signal indicative of lung volume change during breathing with a first sensor;
obtaining a second signal indicative of distending pressure with a second sensor, wherein at least one of the first and second sensors is chronically implanted;
calculating lung compliance and/or pulmonary resistance based on the first signal and the second signal; and
monitoring lung compliance and/or pulmonary resistance values over a period of time to obtain a lung compliance or pulmonary resistance trend.

17. The method of claim 16, further comprising evaluating whether or not the lung compliance or pulmonary resistance trend is adverse.

18. The method of claim 16, further comprising initiating administration of appropriate therapy based on the lung compliance or pulmonary resistance trend.

19. The method of claim 16, further comprising reporting the lung compliance or pulmonary resistance trend to a care provider.

20. An implantable medical device comprising:
a first sensor configured to produce a first signal indicative of lung volume change during breathing;
a second sensor configured to produce a second signal indicative of intrapleural pressure; and
a processor configured to calculate a pulmonary function parameter based on the first signal and the second signal.

21. The implantable medical device of claim 20, the first sensor comprising an impedance sensor.

22. The implantable medical device of claim 20, the second sensor comprising a pressure sensor.

23. The implantable medical device of claim 20, wherein at least one of the first sensor and the second sensor are in wireless communication with the processing unit.

24. The implantable medical device of claim 20, further comprising an electrode configured to stimulate contraction of the diaphragm.

25. A method of titrating drug therapy comprising:
obtaining a first signal indicative of lung volume change during breathing from a first sensor;
obtaining a second signal indicative of distending pressure from a second sensor, wherein at least one of the first and second sensors is chronically implanted;
calculating a value of a pulmonary function parameter based on the first signal and the second signal;
comparing the value of the pulmonary function parameter with a baseline value of the pulmonary function parameter; and
adjusting drug therapy if indicated based on comparison between the value of the pulmonary function parameter and the baseline value of the pulmonary function parameter.
